(19) United States
(12) Patent Application Publication
(10) Pub. No.: US 2008/0167691 A1
(43) Pub. Date: Jul. 10, 2008

(54) Mechanical Defibrillator

(75) Inventor: David Weintraub, Yavne (IL)

Correspondence Address: SOROKER-AGMON ADVOCATE AND PATENT ATTORNEYS, NOLTON HOUSE, 14 SHENKAR STREET, HERZELIYA PITUACH, 46725, omitted

(73) Assignee: David WEINTRAUB, Yavne (IL)

(21) Appl. No.: 11/915,160

(22) PCT Filed: May 29, 2006
(86) PCT No.: PCT/IL06/00630

§ 371 Date: Nov. 21, 2007

(30) Foreign Application Priority Data
May 30, 2005 (IL) 168874

(51) Int. Cl. A61N 1/00

(52) U.S. Cl. 607/5; 600/16

(57) ABSTRACT

A mechanical defibrillator comprising an impact member configured to deliver a mechanical impact of a preset force to the chest of a subject in need of a defibrillation shock.

### BACKGROUND OF THE INVENTION

1. Field of the Invention

The present invention relates to a portable defibrillator, more specifically to a portable rechargeable mechanical defibrillator that uses stored mechanical energy for delivering a mechanical defibrillation impact to a patient's chest.

2. Discussion of the Related Art

One of the main causes for primary sudden cardiac arrest (SCA) is ventricular fibrillation (VF) wheel the electrical system of the heart short circuits, causing the heart to quiver rather than pump in a normal rhythm. As blood is no longer being pumped effectively, a serious brain damage and death may result unless a normal heart rhythm is restored within a few minutes. There are many causes for VF including congenital defects, illness, heart attack and environmental conditions. Although VF is more abundant with elder people, it may also occur in young sports participants as a result of a hard blow to the chest. The phenomena, known as commotio cordis, affects young people with no preexisting heart disease. The bottom line is that VF may occur at any age and anywhere.

Defibrillation, shocking the heart into a normal rhythm, is the only effective way to treat VF. The shock terminates the electrical chaos and allows the hear-t's natural pacemaker areas to restore normal function and resume the normal pumping action of the heart. The chance of surviving sudden cardiac arrest decreases with time. For the best chance of survival, a defibrillation shock should be delivered within a few minutes. A quick response by administering a defibrillating shock as soon as possible is therefore critically important. The American Heart Association estimates that 50,000 lives would be saved every year in the United State alone if equipment for treating VF were more widely available. The importance and essentiality of a widespread of defibrillation equipment is expressed by the Cardiac Arrest Survival Act passed by the US Congress on Oct. 26, 2000. The act provides for placement of Automated External Defibrillator (AED) in all federal public buildings.

The conventional treatment for cardiac arrest is the application of a strong electrical shock to the patient. Defibrillators for producing and delivering electrical shocks have been known and successfully used for many years in hospitals and emergency medical facilities. Recently, technological progress has led to the development of Automated External Defibrillators (AED) for delivering electric shock that are designed to be used by lay rescuers in an emergency when medical professionals are not present. However, the price of these devices is still high, hindering wide spread in public places and private accommodations. Furthermore, defibrillators for delivering electrical shocks rely on electric power for producing the electrical shock. The electrical power can be supplied from an electric power, in this case the use of the device is limited to places where such a line is available. Alternatively, and more commonly in mobile AEDs, the power source is a battery. However, batteries have a limited power that suffices to a limited number of electrical shocks and need to be recharged or replaced before the device can be reused. Moreover, batteries deteriorate with time losing their power. Thus, a defibrillator that is not properly maintained may fail to operate in time of need. Another main drawback associated with external electric defibrillators is the risk of electric shock to the device operator or to other persons in the patient's vicinity. Thus special precautions should be taken in order to prevent any contact with the patient.

Another method for shocking a fibrillating or a standstill heart to resume its normal rhythm is by a mechanical shock. It is known that one or two blows to the mid chest given with a clenched fist can restart the heart spontaneously, obviating the need for chest compression. This method, known as ‘the chest thump’ or “the pericardial thump” is recommended by some organizations as part of cardiac emergency procedure. Although this method has been proven to be life saving, it has its drawbacks, mainly the inability to control the intensity of the thump blow in a reproducible manner. A too powerful blow may cause physical harm while a weak blow may be ineffective. Thus, in order to facilitate the practice of this useful life-saving procedure, especially where electrical AED's are not available, there is a need to establish a standardized procedure and to provide a means that will enable the delivery a mechanical impact of a preset value to the sternum of a cardiac arrest victim in a controlled manner, so that the force and duration of the impact is independent of the specific individual attending the victim. Accordingly, it is the general object of the present invention to provide a mechanical defibrillator adapted for delivering a mechanical impact of a preset value to a patient chest.

It is another object of the invention to provide a mechanical defibrillator that can be manually charged by the operator prior to delivering the mechanical blow.

It is a further object of the invention to provide a mechanical defibrillator that does not rely on electrical power source, that is easy to operate, is lightweight, low-cost, durable and reliable.

It is another object of the invention to provide a mechanical defibrillator that receives its energy from a mechanical energy storage element that can be manually charged to a predetermined level by a simple manual operation.

Yet a further object of the invention is to provide a mechanical defibrillator that can be operated by people with minimal medical training in an emergency.

A further object of the invention is to provide a mechanical defibrillator that is having a sensing means for recognizing ventricular fibrillation (VF) and means to prevent operation of the device if VF is not detected

These and other objects of the invention will be apparent to those skilled in the art from the following description.

### SUMMARY OF THE PRESENT INVENTION

The present invention provides a mechanical defibrillator configured to deliver a controllable mechanical impact of a preset force to the chest of a cardiac arrest victim. The impact force may be of a preset fixed value or of a preset adjustable value. The defibrillator comprises an impact member mounted on a frame adapted to be placed on the victim's chest. The defibrillator further comprises a rechargeable energy storage element coupled to the impact member and configured to be charged when the impact member is elevated above the frame and to discharge when the impact member is released, whereby the energy discharged from the rechargeable element is transferred into the mechanical impact. The amount of energy charged into the chargeable element corresponds to the height to which the impact member is elevated. The mechanical defibrillator further comprises a controllable discharging mechanism for releasing energy stored in the rechargeable energy storage element into impact energy of the impact member and a controllable charging mechanism for controllably elevating the impact member to a controllable height above the subject's chest. The charging mechanism is preferably operated manually by the defibrillator's operator. Preferably, the device can be controllably charged to deliver an impact of up to 180 Kg. The defibrillator may further comprise an indicator for indicating the charging level. The impact member is preferably a cylindrical weight of about 100-400 gr and of about 20-50 mm diameter.

The defibrillator may be further provided with at least one sensor for sensing heart's activity of the subject and with analyzing means for analyzing the heart activity and to detect ventricular fibrillation. The defibrillator may further comprise halting means to prevent discharge if ventricular fibrillation is not detected.

The defibrillator may further comprise activating elements for manually activating said discharging mechanism. The activating elements may comprise a discharger element configured to release the impact member, a stopper element configured to prevent the discharging member from discharging the impact element and a stopper releasing element configured to release the stopper element for enabling discharging. The defibrillator may further comprise a main stopper configured to prevent discharging unless the defibrillator is pressed properly against the subject's chest.

The invention further provides a method for delivering a defibrillation shock to a cardiac arrest victim, the method comprising the step of delivering a controllable mechanical impact of a preset force to the chest of the victim by means of a mechanical device provided with an impact member.

### BRIEF DESCRIPTION OF THE DRAWINGS

The present invention will be understood and appreciated more fully from the following detailed description taken in conjunction with the drawings in which:

FIGS. 1 and 2 are isometric views of a mechanical defibrillator in accordance with a first embodiment of the present invention, showing the defibrillator in unloaded state and in a loaded, state, respectively;

FIG. 3 is an isometric bottom of the defibrillator of FIG. 1;

FIG. 4 is a top view of the defibrillator of FIG. 1 with top cover removed to show internal structure;

FIG. 5 is a partial view of a the defibrillator of FIG. 1 demonstrating the charging mechanism of the device;

FIGS. 6 and 7 are isometric views taken from opposite directions of the device of FIG. 1 in an upside down position and with external cover and electronic elements removed for demonstrating the discharge elements; FIGS. 6A and 7A demonstrate the elements in their default positions; FIGS. 6B and 7B demonstrate the elements in their activated position;

FIG. 8 is an isometric view taken from the same direction as of FIG. 6 demonstrating the electronic components of the device of FIG. 1;

FIGS. 9 and 10 are isometric top view and bottom view, respectively, of a mechanical defibrillator in accordance with a second embodiment of the present invention;

FIG. 11 is a bottom view of the defibrillator of FIG. 9 with bottom cover removed showing the defibrillator in an unloaded state;

FIG. 11A is an exploded view of the torque spring of the embodiment of FIGS. 9-13;

FIG. 12 is a bottom perspective view of the embodiment of FIG. 9 with bottom cover removed showing the defibrillator in a loaded state;

FIG. 13 is a top perspective view of the defibrillator of FIG. 9 in a loaded state;

FIGS. 14 and 15 are a top view and a bottom perspective view, respectively, of a defibrillator in accordance with a third embodiment of the invention;

FIGS. 16A and 16B are a front and a rear isometric view of a spring force measuring device;

### DETAILED DESCRIPTION OF THE PREFERRED EMBODIMENTS

The present invention provides a mechanical defibrillator configured for delivering a mechanical impact of a predetermined and reproducible power to the chest of a cardiac arrest victim. The device comprises an impact member mounted on a frame which is configured to be placed on a patient chest. The impact member is coupled to a rechargeable energy storage element. A controllable means to release energy stored in the energy storage element allows for activating the impact member to deliver a blow of a predetermined power to the patient chest. Preferably, the energy storage element is charged manually prior to the impact delivery. The defibrillator of the invention may be designed to deliver an impact of a preset fixed power or may be designed to allow for the adjustment of the impact power within a predetermined range. Preferably, the impact force is of up to 180 Kg, more preferably the force is in the range of 60-160 Kg and of about 10-50 msec duration.

As will be demonstrated by the following embodiments, the power supply for the defibrillator of the invention is preferably a rechargeable mechanical energy storage element that may charged manually by the device operator immediately prior to delivering a blow. This renders the device the advantage of inexhaustible power. Additionally, since the device has to be charged deliberately prior to each blow, it has the advantage of reducing the chance of an accidental blow. Further in order to prevent accidentally delivered blows, the device may be provided with a number of independent precautionary stoppers and corresponding activation triggers for ensuring that a blow cannot be delivered unless the device is appropriately placed on the patient's chest and/or the patient is in a condition for a defibrillation shock.

Being independent of external energy source and manually rechargeable enhances the portability of the device as well as the low cost of maintenance and use. The device can be used anywhere, at home and in public places, and can be provided as a component in any first aid kit.

Although, charging the device is preferably performed manually, it will be understood that the present invention is not limited to manual charging mechanism and that other methods may be used for charging the device. For example, a relatively low power motor of small dimension may be used for charging the mechanical energy storage element of the defibrillator to the desired level.

The mechanical defibrillator of the invention may also comprise a sensing means for sensing the heart activity of the patient and computing means for analyzing the heart activity in order to recognize the presence or absence of ventricular fibrillation for determining if a defibrillation shock is appropriate. Analyzing the heart activity may be performed in accordance with analyzing methods known in the art. The device may be further provided with an indicator, such as a LED, for indicating to the defibrillator operator whether the patient heart condition is in a fibrillation condition. After delivering one impact, the sensing means may monitor the heart activity again to check if normal heartbeat has reestablished. A second blow might be delivered if the first shock failed to resume normal rhythm. Optionally, the device is further provided with a halting means for automatically disabling the device from delivering a mechanical shock when the patient's condition, as found by the heart activity analysis, is not suitable for such a shock. It will be realized that the power source required for powering the sensing means and electronic circuit, may be low power battery. Alternatively, or additionally, the device may be provided with an electric capacitor which can be charged manually for supplying the low power needed for the electronic components.

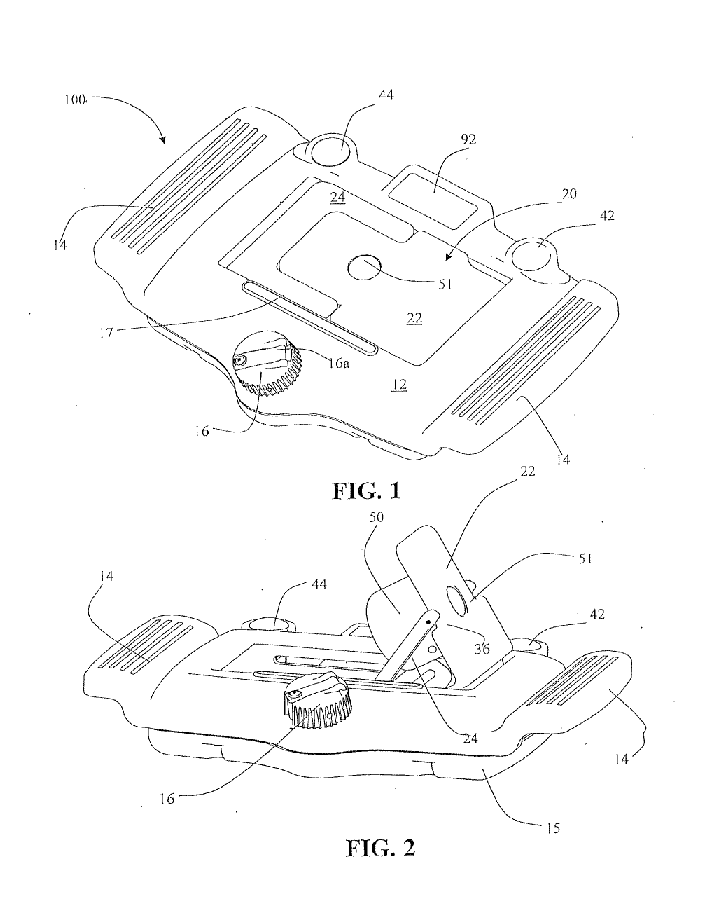

Turning now to the drawings, FIGS. 1-8 illustrate a mechanical defibrillator, generally designated 100, in accordance with a first preferred embodiment of the invention. Referring to FIGS. 1 and 2, defibrillator 100 is having a substantially flat rectangular box-like shape with two handles 14 extending from opposite ends thereof for allowing a firm grip of the device by both hands of the device operator. Preferably the dimensions of the device are of about 220×130×40 mm. The upper face of defibrillator 100 comprises a frame-like top cover 12 of which extending handles 14 are an integral part. Mounted within frame 12 is a two part folding inner door 20 comprising complementary wings 22 and 24. Wing 22 is connected to an impact weight 50 mounted inside device 100, as will explained in detail below, by means of a plug 51 inserted into a corresponding recess at the upper surface of weight 50. Upon charging, door 20, pushed by impact weight 50, folds upwardly as demonstrated in FIG. 2. Wings 22 and 24 are configured so as to allow weight 50 to elevate to maximal position. It should be noted that in accordance with embodiment 100, wings 22 and 24 are not part of the power system of the device but only serve as a cover to protect the inner parts of the device when not in use.

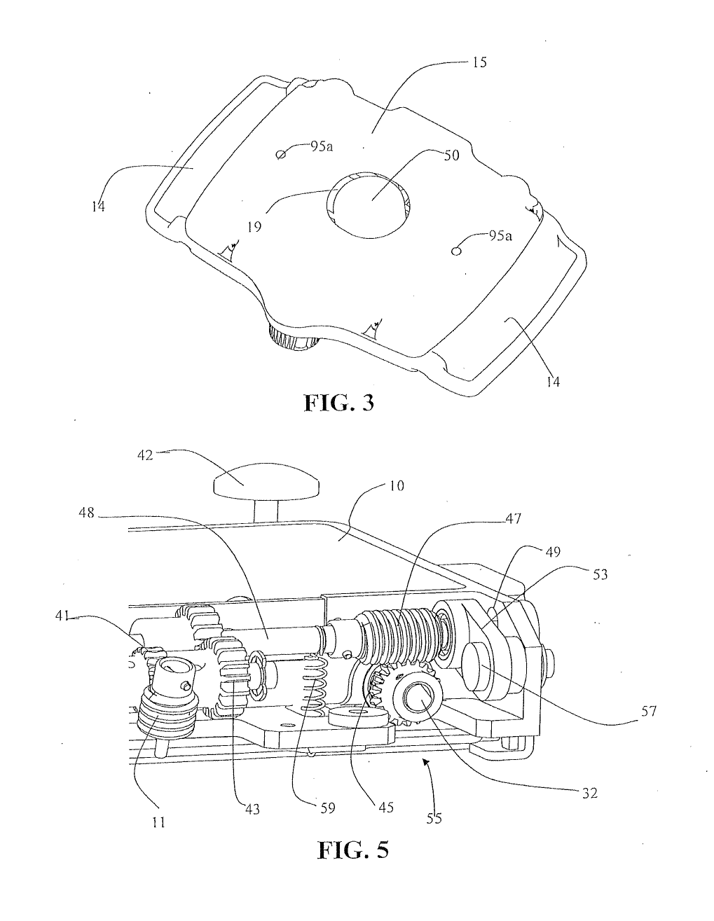

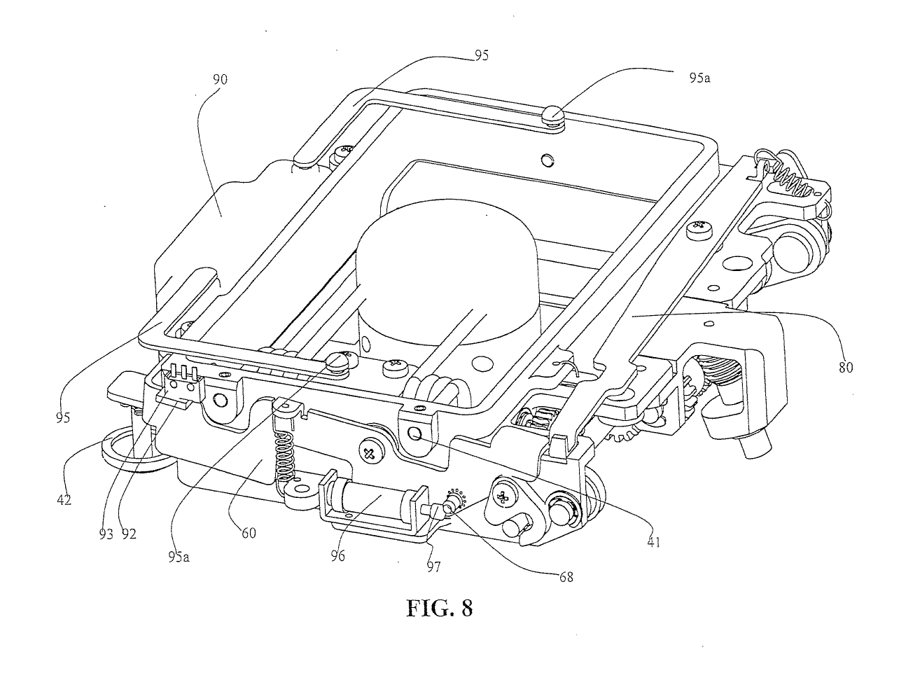

A swivel knob 16 for manually charging the device is provided oil top cover 12. A pullout handle 16a that can be pulled upwardly to a vertical orientation facilitates turning knob 16 around. Also seen on the top of defibrillator 100 are a see-through window 17 for indicating charging level, an electronic display 92 for displaying the patient's heart condition and two push buttons 42 and 44 for activating the device to deliver a blow. Referring to FIG. 3, the bottom cover 15 of defibrillator 100 is having a central opening 19 through which impact weight 50 can protrude. Also seen at the bottom side are the extending tips 95a of two electrodes 95 (seen in FIG. 8). In operation, device 100, after being charged to a desired level read through window 17, by manually turning knob 16, is placed on the patient's chest to form a contact with electrodes 95. If the patient's heart condition, as sensed by electrodes 95, is suitable for defibrillation shock, then while firmly holding device 100 by both hands with fingers bent under handles 14 and pressing the device against the patient's chest, push buttons 42 and 44 are pressed down by the operator's thumbs, discharging impact weight 50 to deliver a mechanical blow through opening 19.

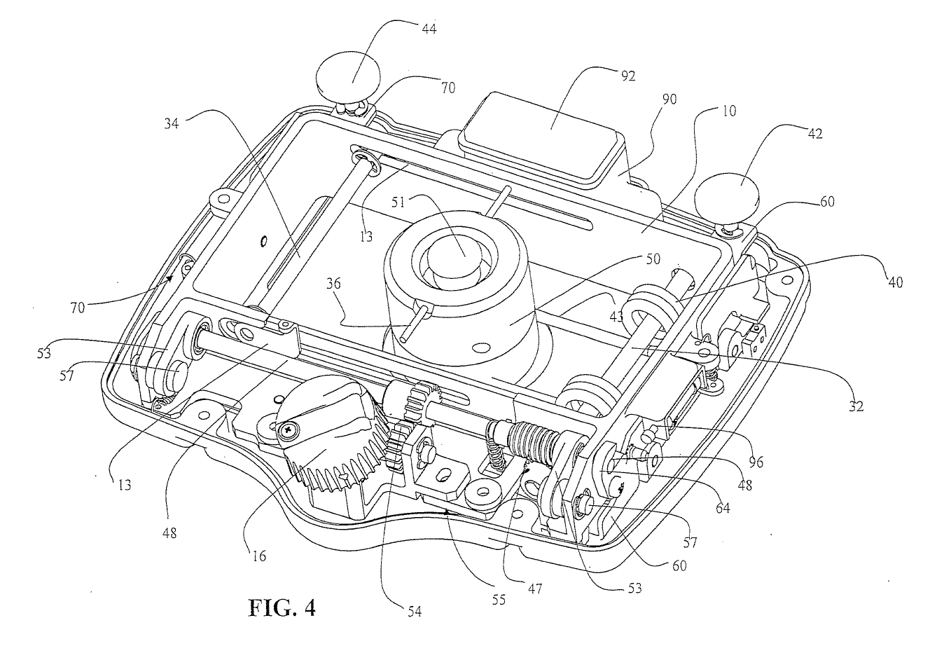

Referring to FIG. 4, an overall view of the internal structure of device 10 is shown. The internal components as well as the outer cover parts are all mounted on a substantially rectangular chassis 10. This allows for a strong construction of the device and the ability to withstand the impact shock delivered by impact weight 50.

The two wings 22 and 24 of folding door 20 described above are mounted around axis 32 and 34, respectively, extending between opposite walls of chassis 10 and around common two-part axis 36 fixedly inserted into weight 50. Axis 32 is fixedly connected to opposite walls of chassis 10 while axis 34 is slideably mounted between opposite elongated slits 13, allowing cover 20 to fold upwardly around axis 36 when weight 50 is charged, as seen in FIG. 2. As mentioned above, wing 22 of door 20 is connected to weight 50 by means of plug 51.

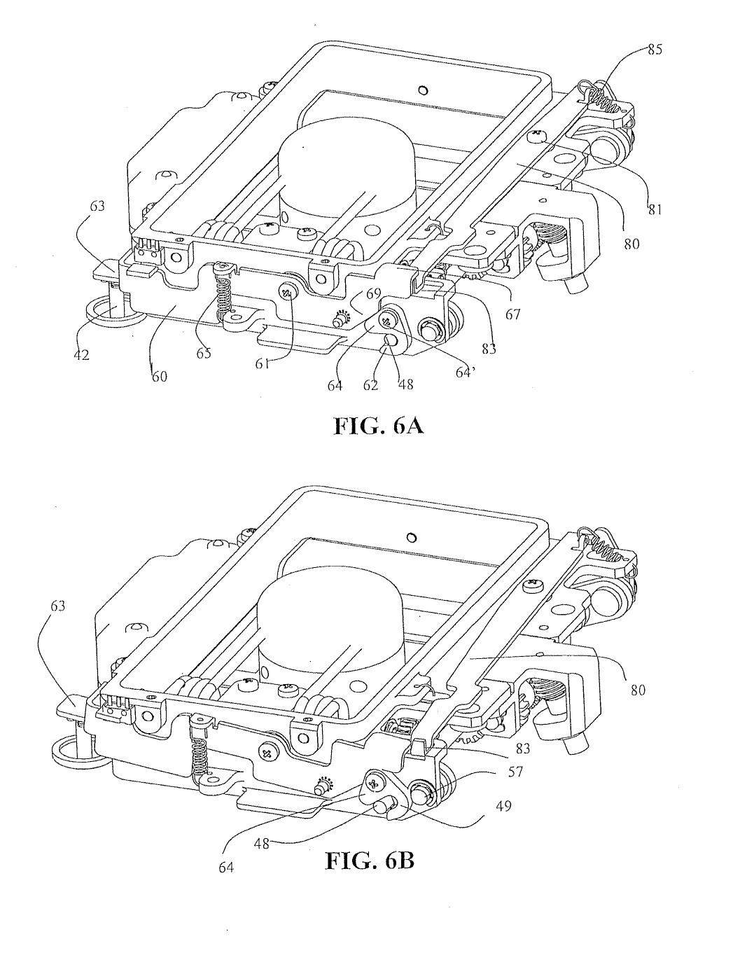

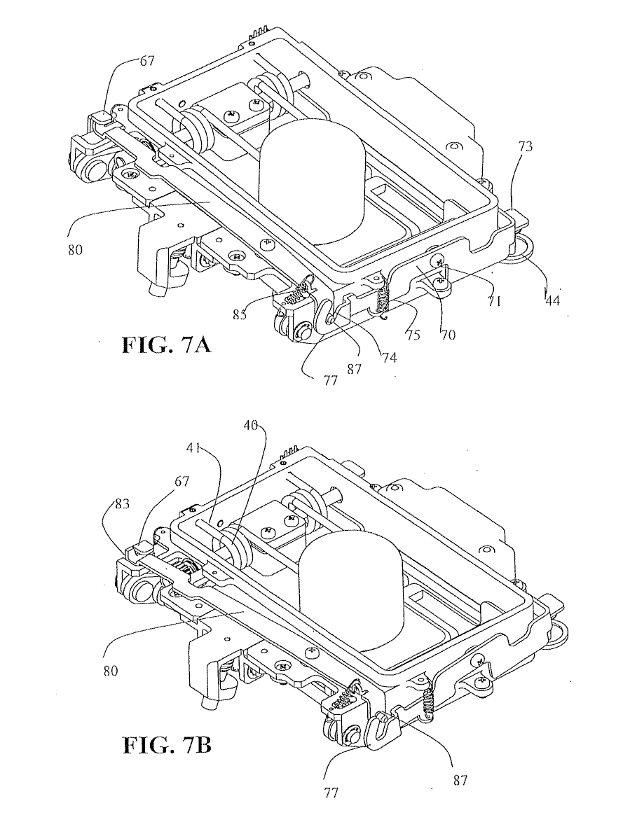

The internal components of device 100 can be grouped into the following sub-systems: a power system including impact weight 50 mounted on axis 32 in a mouse-trap manner by means of torque springs 40; a charging system, generally designated 55, for manually charging springs 40 via knob 16; a discharging system (best seen in FIGS. 6 and 7) comprising a discharge member 60 coupled to push button 42, a stopper member 80 and a stopper releasing member 70 coupled to push button 44; and an electronic system comprising electrodes 95 and solenoid 96 both connected to electronic circuit housed in electronic box 90.

Torque springs 40 comprise a coiled portion of a few loops coiled around axis 32 terminating with two arms 41 and 43. Longer arm 43 is fixedly connected to weight 50. The second arm 41 (best seen in FIGS. 7 and 8) is fixedly connected to chassis 10. Weight 50 is preferably a cylinder of about 40 mm diameter, 40 mm height and 300 g weight, made of rigid durable plastic material or coated metal. The bottom face of weight 50 is slightly rounded and preferably coated by a thin layer of soft layer to cushion the impact and to prevent injury. Springs 40 are preferably 3 mm stainless steel springs wherein the length of arm 43 is preferably of about 50 mm. Preferably, the equilibrium state of springs 40 is at an angle of about −20 deg. with respect to the plane of chassis 10 (namely, directed downwardly by 20 deg.) and the maximum angle corresponding to maximum charging by is of about 60-80 deg. above the chassis plane. A pointer 13 mounted at one end of moving axis 34, in alignment with window 17, indicates through the window the extent to which axis 34 travels, hence the extent to which springs 40 are loaded.

Turning to FIG. 5, there is shown in detail the charging mechanism 55 for charging springs 40. Axis 48 is coupled to worm gear 11 of knob 16, via spur gears 41 and 43. Axis 32, around which torque springs 40 are coiled (see FIG. 4), is coupled to axis 48 via another worm-spur gear couple 47-45. Thus, by turning knob 16 around its axis, the rotational movement is transferred to axis 32 to load springs 40. It should be noted that the two worm-spur gears' namely, 11-41 and 47-45, allow for two-fold force reduction, for facilitating manually charging springs 40 to high torque levels with a relative ease. It should also be noted, that the structure or gear 47-45 prevents axis 32 from rotating under the torque exerted on axis 32 by loaded springs 40. As best seen in FIG. 4, axis 48 is mounted between two pivoting arms 53 pivotally mounted about pivoting axes 57. One end of axis 48 is protruding through opening 49 in chassis 10 dimensioned to allow a limited rotation of arms 53 around pivots 57. In order to prevent possible accidental disengagement between gears 47 and 45, a hook like pawl 64 (best seen in FIGS. 4 and 6) prevents arms 53 from rotating, retaining gears 47 and 45 engaged. Additionally, a biasing spring 59 fixedly connected at one end to chassis 10 keeps the gears engaged by pulling axis 48 downwardly. As explained below, discharger element 60 is arranged to disengage gears 47 and 45 by pressing down button 42, thus releasing axis 32 to freely rotate under springs 40 torque, discharging the energy stored in the springs.

FIGS. 6 and 7 are two isometric bottom views of device 100 with external covers and electronic component (solenoid and electrodes) removed to demonstrate the discharging mechanism. The discharging mechanism comprises member 60 pivotally mounted on one lateral wall of chassis 10 around pivot 61; member 70 pivotally mounted on the opposite lateral wall of chassis 10 around pivot 71; and member 80 pivotally mounted on the bottom side of chassis 10 around pivot 81. Members 60, 70 and 80 are each configured to shift between a default position and an activated position triggered by push buttons 42 and 44. Members 60, 70 and 80 are configured so that in order to shift them to their activated positions, both buttons 42 and 44 should be pressed simultaneously. Members 60, 70 and 80 are kept at their default positions, shown in FIGS. 6A and 7A, by biasing springs 65, 75 and 85, respectively. As mentioned above, discharging loaded springs 40 is performed by disengaging axis 32 from axis 48. Referring to FIG. 6, axis 48 is extending through opening 49 in chassis 10. Opening 49 is dimensioned to allow a small limited range of rotation of axis 48 around pivot 57 as described above in association with FIG. 5. A pawl 64 pivotally mounted to chassis 10 around pivot 64′ is configured to retain axis 48 in its place engaged with axis 32 by means of hook 62. Slope 69 of discharger member 60 is configured to tilt pawl 64 and consequently pushing axis 48 away from axis 32 to the position shown in FIG. 6B when member 60 is tilted around pivot 61 by means of button 42. However, vertical extension 83 protruding upwardly from one end of member 80 and located under horizontal extension 67 of member 60 when member 80 is in its default position, prevents member 60 from tilting. Referring to FIG. 7, the second end of member 80 is provided with an extension 87 engaged with hook-like end 77 of member 70 when member 70 is in its default position. Referring to FIGS. 7B and 6B, upon pressing button 44, hook 77 of member 70 is tilted downwardly, thereby extension 87 is pushed backwardly by slope 74 of hook 77. Consequently, member 80 is tilted to shift stopping protrusion 83 from its location under extension 67. Thus, if button 42 is pressed at the same time, discharging member 60 is free to shift, thereby the frontal end of member 60 disengage axis 32 from axis 48 allowing axis 32 to rotate under the torque of springs 40. It will be realized that as soon as buttons 42 and 44 are released, members 60′ 70 and 80 return to their default position by means of springs 65, 75 and 85 and axis 48 returns to engaging position with axis 32 by means of spring 59 (seen in FIG. 5). However, buttons 42 and 44 can be kept pressed after a blow has already been delivered with no effect on the power system.

FIG. 8 depicts the electronic components of device 100 that include electrodes 95 and solenoid 96, both connected, to electronic circuit (not shown) housed in electronic compartment 90. The electronic circuit includes a microprocessor for analyzing the signals received from electrodes 95 in accordance with methods known in the art for determining the patient's heart condition and in particular to determine whether a fibrillation shock is appropriate. The electronic circuit further includes control means to enable/prevent delivery of a shock in accordance with the analysis results. The electrocardiogram may be displayed on display 92. Alternatively, or additionally, the analysis results may be displayed on display 92 as well as instructions regarding operation of the device. Optionally, or additionally, the electronic instructions may be voice instructions. An indicator, such as a LED may be further provided for indicating whether or not to activate the device. The electronic control of the mechanical discharge mechanism described above is performed by means of a pull solenoid 96 connected to the electronic circuit via switch 93. A rod 68 protruding from member 60 is configured such as to prevent tilting of member 60 when plunger 97 is in its extended position, i.e., when no current is supplied to solenoid 96, and to enable such tilting when solenoid 96 receives current to draw plunger 97 inwardly. Thus, activation of the mechanical discharging elements is possible only when solenoid 96 is powered. The power supply to solenoid 96 is controlled by the electronic circuit via switch 93 in accordance with the electrocardiogram analysis. If ventricular fibrillation is recognized, solenoid 96 is powered and buttons 42 and 44 can be pressed down to deliver a blow. At the same time, switch 93 is pressed by extension 94 of member 60. Solenoid 96 is kept powered as long as button 42 is pressed down, as indicated by switch 93, in order to prevent possible damage to plunger 97 from being released to its extended position before member 60 resumes its default position. It will be realized that the electronic control of the discharge mechanism not only prevents firing a mechanical shock when the patient's heart condition is not suitable for such a shock but also prevents misuse of the device for other than defibrillation purposes.

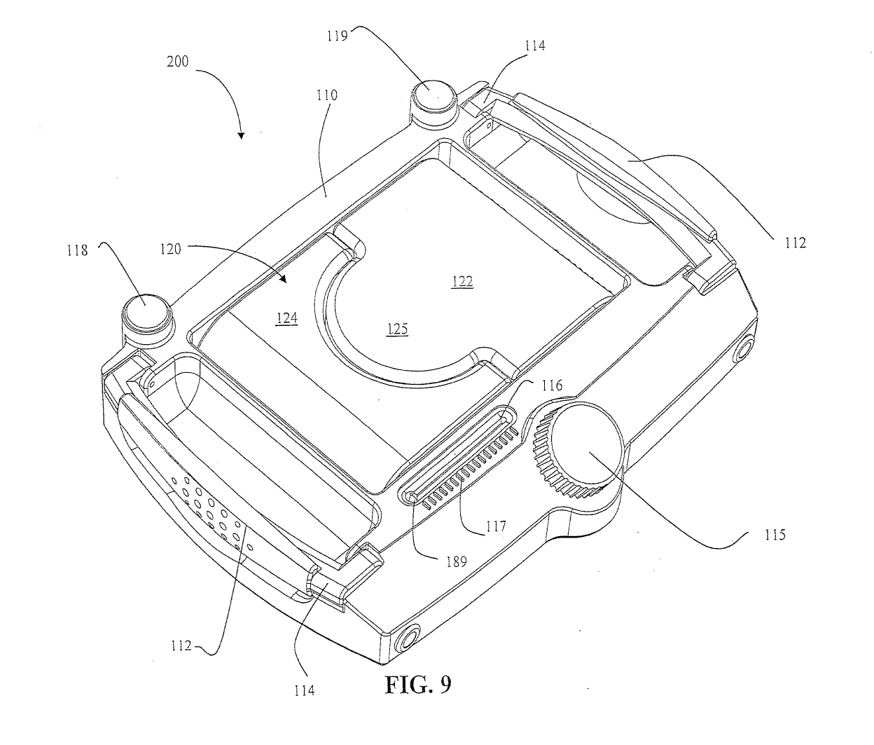

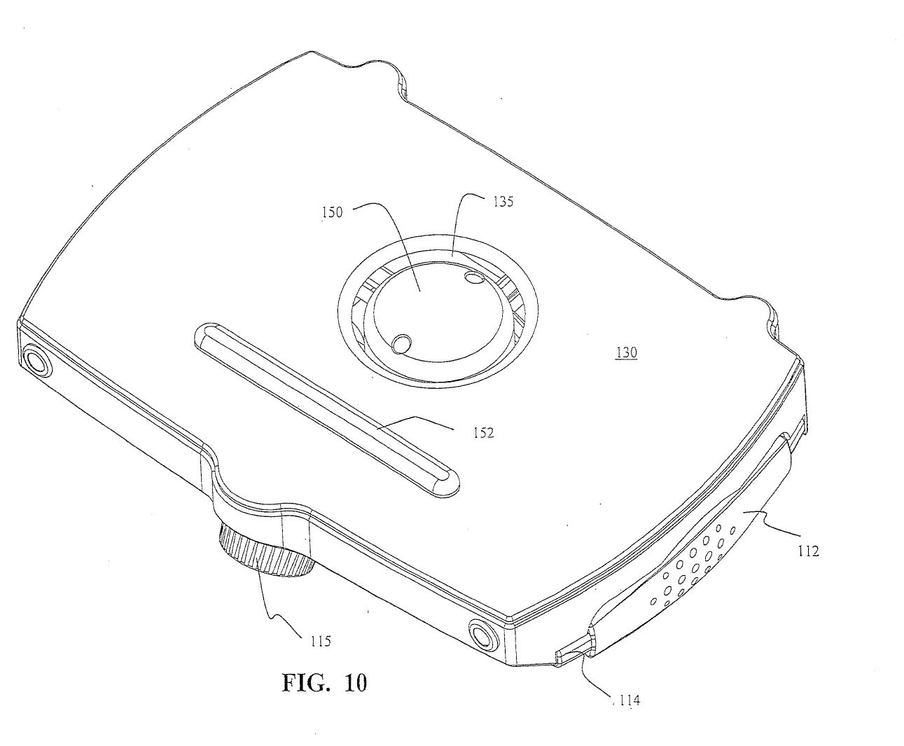

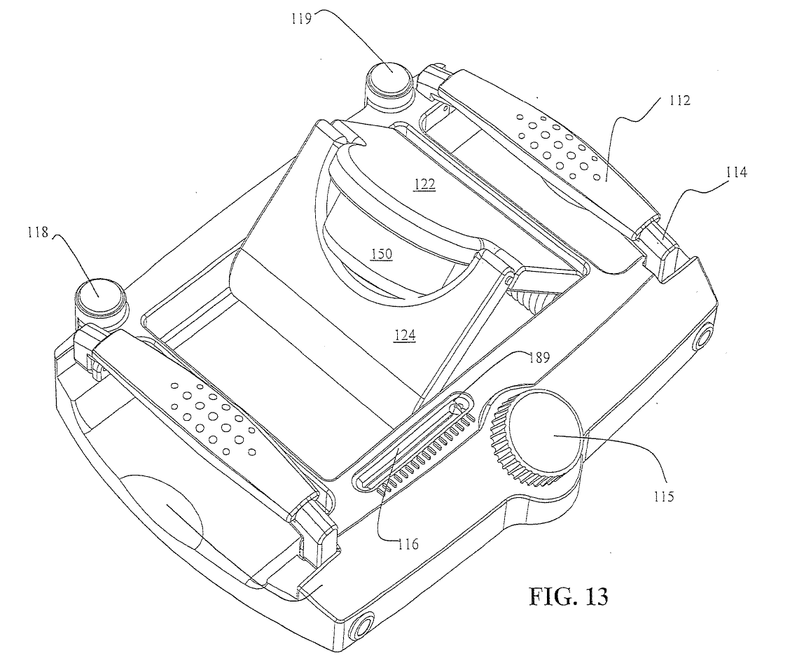

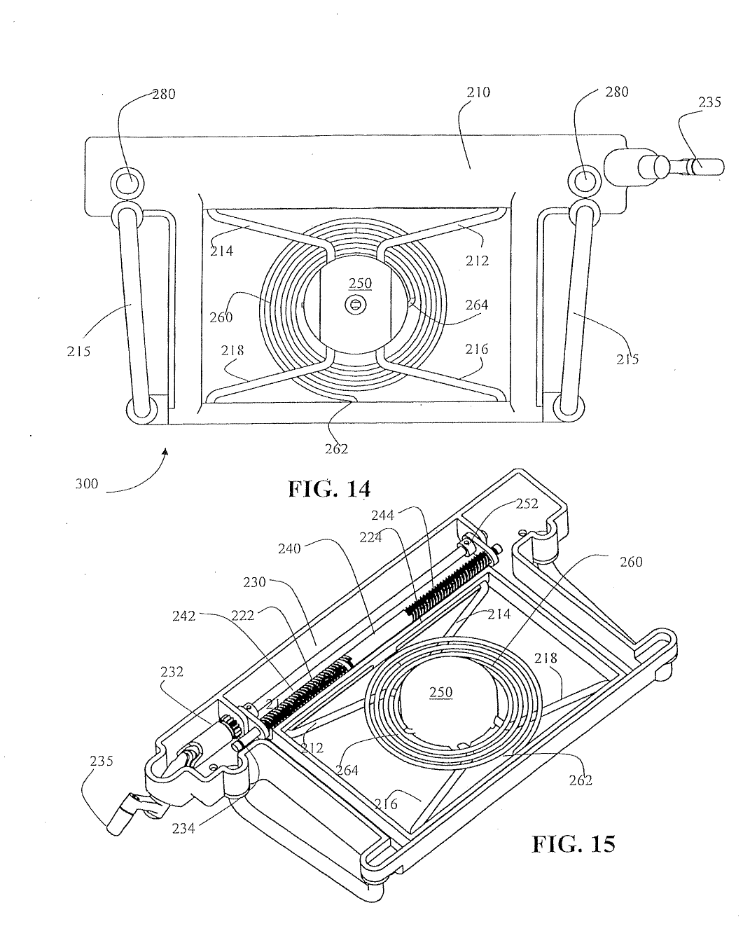

FIGS. 9-13 depict a second embodiment of a mechanical defibrillator of the invention, generally designated 200. The dimensions of embodiment 200 are similar to those of embodiment 100. Defibrillator 200 comprises a substantially rectangular frame 110 having a plurality of reinforcing partition ribs for accommodating and supporting the various components of the device. Two handles 112, supported on pivoting arms 114 are located on opposite lateral ends of frame 110. Pivoting arms 114 allow for laying handles 112 in a storage position when not in use as is seen in FIG. 9 and to rotate handles 112 upwardly to assume a vertical position as is seen FIG. 13. Frame 110 is provided on its frontal surface with a swivel knob 115 for manually charging the device as will be explained below and a see-through scale window 116 with adjacent graduations 117 for indicating the energy level to which the device is charged and corresponding power of delivered impact. The scale may be further provided with figure images such as a child figure, an adult figure etc., for facilitating the operator to select the power suitable to the patient in hand. Also provided on the frontal face of frame 110 are two spring-loaded press buttons 118 and 119 configured to be pressed down by the operator thumbs in order to deliver an impact while holding handles 112 and pressing the device against the patient's chest. In accordance with embodiment 200, an additional independent stopper 152 (see FIG. 10), provided at the bottom of device 200, prevents discharging the device unless the device is properly presses against the patient's chest.

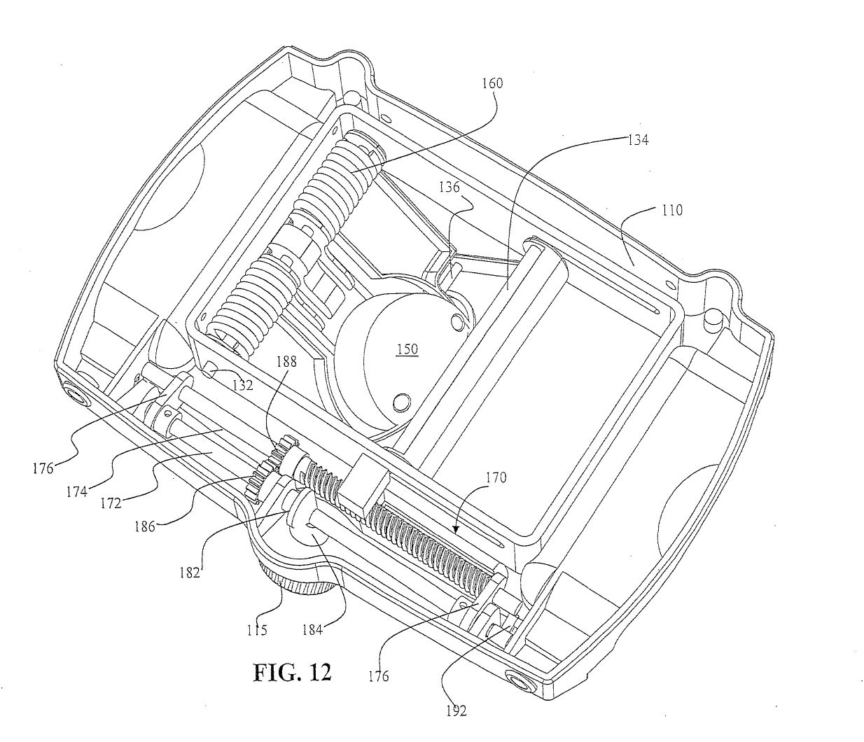

A two-part folding upper cover 120, comprising of two complementary wings 122 and 124, is mounted within the upper opening of frame 110. Wings 122 and 124 are hingedly mounted around common axes 136a and 136b at their proximal ends and around fixed axis 132 and movable axis 134 at their distal ends, respectively, in a similar way to wings 22 and 24 of embodiment 100 above. Axis 132 is fixedly connected to opposite inner walls 121 of frame 110. Axis 134 is slideably mounted on opposite elongated slits 142a 142b between opposite walls 121 to allow cover 120 to fold outwardly around axis 136 as axis 134 slides inwardly toward fixed axis 132 (as shown in FIGS. 12 and 13). An impact member 150 is fixedly mounted on the inner face of semicircular extension 125 of wing 124 substantially at the center of frame 110 when cover 120 is in a flattened position. Member 150 is preferably a rounded-bottom cylinder of about 400 nm diameter made of rigid durable plastic material or light weight metal. Wings 122 and 124 are configured to allow the folding of cover 120 to maximum upward position with no hindrance by member 150. Cover plate 130 having an opening 135 through which weight 150 protrudes, closes the bottom face of frame 110. Also provided on plate 130 is a main stopper 152.

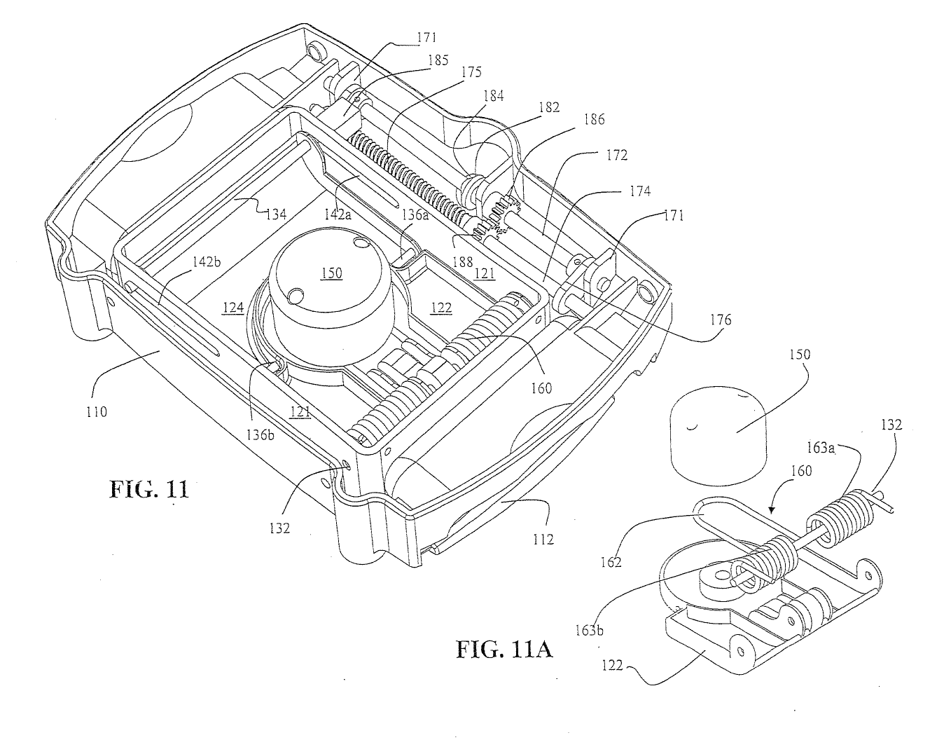

Turning now to FIGS. 11 and 12, the charging mechanism for loading weight 150 is depicted. In accordance with embodiment 200 the chargeable element is a coil spring 160 comprising a U-shaped lever midportion 162 disposed between two coiling end portions 163a,163b coiled around fixed axis 132 and fixedly connected to frame 110, as best seen in FIG. 11A. The closed end of lever 162 is confined between weight 150 and wing 122. It will be realized folding cover 120 around axis 136 generates a torsion force to load spring 160 in a mousetrap-like manner, as seen in FIGS. 12 and 13. The charging mechanism responsible for charging spring 160 via knob 115 is accommodated within inner compartment 170 in frame 110. The mechanism includes axis 172 rotatably mounted between supporting ribs 171 and a parallel shaft 174, having worm portion 175, connected to axis 172 by arms 176. Arms 176 retain the relative position of shaft 174 and axis 172 but allow for rotation of shaft 174 about axis 172. A bevel 182 mounted on axis 172 is constantly engaged with inner bevel 184 of swivel knob 115 such that turning knob 115 causes axis 172 to turn around itself. Engaging gears 186 and 188 of axis 172 and shaft 174, respectively, couples between the rotational movement of axis 172 and shaft 174. A rider-bar 185 extending from moving axis 134 and engaged with worm portion 175 of shaft 174 couples between shaft 174 and moving axis 134. The inner face of bar 185 (not shown) is threaded to match worm 175 so that as shaft 174 is turning around, bar 185 travels inwardly along worm 175. Thus, when knob 115 is turned around, it causes axis 172 to rotate about itself, which in turn causes shaft 174 to rotate in opposite direction via engaging gears 186, 88. As shaft 174 rotates about its axis, rider-bar 185 travels along shaft 174 forcing wings 122 and 124 to fold outwardly away from bottom cover 130 (and away from the patient chest), loading mainspring 160. A pointer 189 (seen in FIG. 13) extending from rider-bar 185 toward cover 120 and aligned with window 116 indicates through the window the extent to which slide-bar 185 travels, hence the extent to which spring 160 has been loaded. Two stoppers 192 press shaft 174 against rider-bar 185, preventing shaft 174 from moving under the force exerted on the rider by spring 160 as the spring is loaded. Stoppers 192, coupled to main stopper 152, are configured to rotate away from shaft 174 when stopper 152 is pressed down. Embodiment 200 is further provided with a discharging mechanism (not shown) comprising a discharge element, a stopper element and a stopper-releasing element, coupled to buttons 118 and 119 which may be constructed in a similar way to the discharging mechanism of embodiment 100 above. Thus, in accordance with embodiment 200, both buttons 118 and 119 as well as stopper 152 should be all pressed at the same time in order to disengage shaft 174 from rider bar 185, thereby lever 162 is released to swing downwardly under the spring force, pushing impact member 150 to deliver a blow. At the end of the blow, cover 120 resumes a flatten position, rider 185 resumes its position at the far end of worm 175 and the device is ready to be recharged.

FIGS. 14 and 15 depict yet a further embodiment of a mechanical defibrillator, generally designated 300. In accordance with this embodiment, the impact member 250 is mounted on four arms 212, 214, 216, and 218 slideably mounted in slits 222, 224 in one inner wall and opposite slits (not shown) in opposite wall of frame 210. A spring 260 supported on arms 212, 214, 216, 218 around impact member 250 is fixedly connected at one end 262 to frame 210 and at the second end 264 to member 250. When arms 212, 214, 216, 218 slide inwardly, impact member 250 is pushed upwardly (i.e., away from the patient chest) while at the same time spring 260 is loaded. Arms 212 and 214 are coupled to mirror worm portions 242 and 244 of shaft 240 by means of rider bars (not shown) extending from the arms into compartment 270 configured to travel inwardly toward each other as the shaft rotates around its axis in a similar manner as rider-bar 185 travels along shaft 174 in embodiment 200 above. An external swivel handle 235 extending from axis 230 is configured to rotate shaft 240 by means of engaging gears 232, 234. Shaft 240 is pivotally connected to axis 230 by arms 252 and is held in position by means of stoppers that prevent the shaft from moving under the force of spring 260. Similarly, to the manner described above in conjunction with embodiments 100 and 200, pressing down spring-loaded triggers 280 rotate the stoppers away from shaft 240 enabling discharge of spring 260 pushing impact member 250 downwardly under the spring torque to deliver a blow directed at the patient's chest. After the blow is delivered, arms 212, 214, 216 and 218 resume their original position and the device is ready to be recharged.

It will be appreciated by persons skilled in the art that the embodiments described hereinabove are given as non-limiting examples of the present invention and that other embodiments are possible without departing from the scope of the invention. For example, the mechanical defibrillator of the invention may not necessarily include a force adjustment mechanism but rather may be designed to be loaded to a fixed preset energy value in order to deliver an impact of a fixed preset value. Likewise, the charging mechanism of the device may be a simpler mousetrap-like mechanism where the impact weight is directly elevated by a pulling movement rather than by a gear system as demonstrated above. Similarly, the discharge mechanism may be designed to be a floating floor that releases the impact weight when the device is pressed down against the patient chest. It will be also appreciated that any of these alternative mechanisms as well as any of the different features described in conjunction with a particular embodiment, may be combined in the design of a device of the present invention.

### A Force Measuring Device

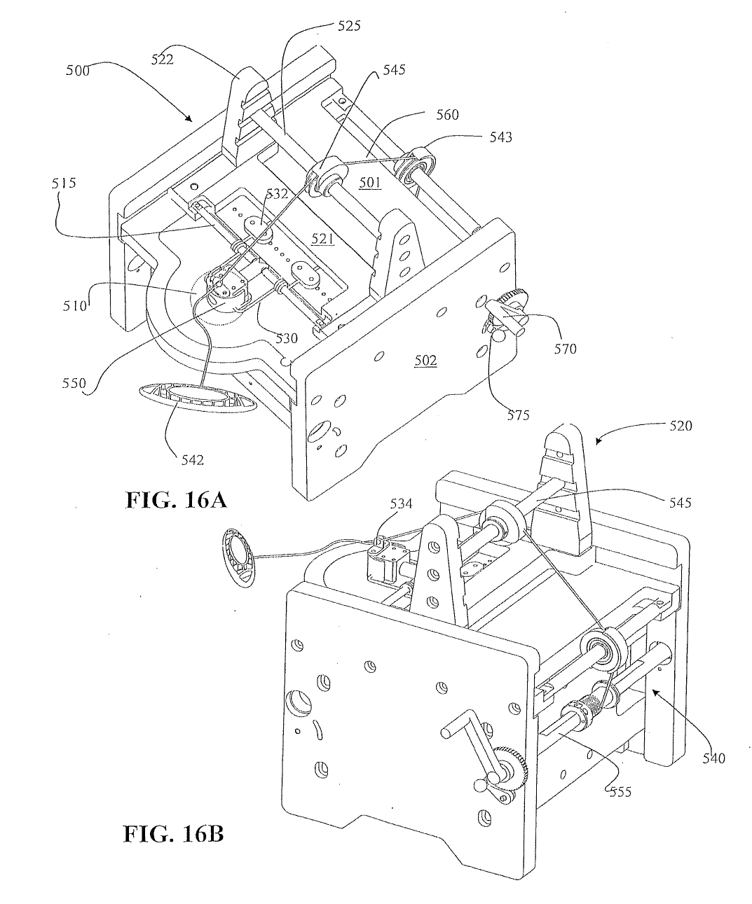

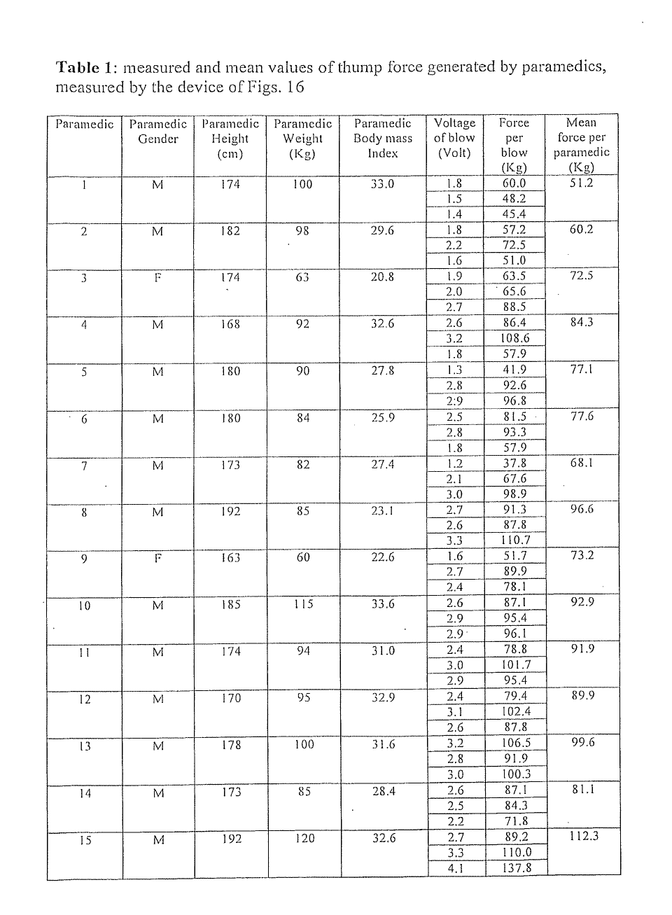

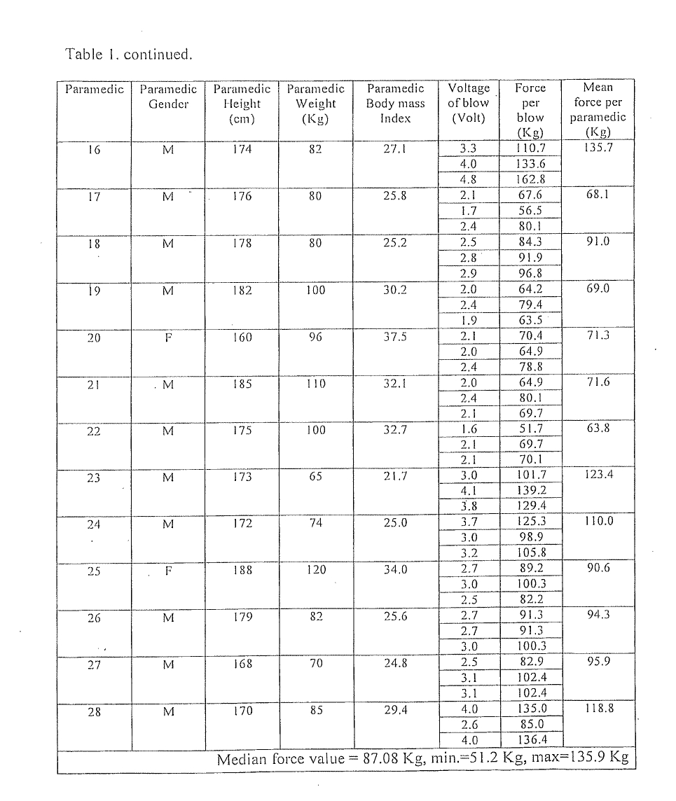

A force measuring device was especially built for measuring the force applied by a blow on an horizontal surface. The device was used to measure the force applied by fist blows of different adults and by an impact of a weight charged by a loaded spring in order to establish values of typical fist blows and to simulate such blows by the mechanical defibrillator device of the invention. The device was designed to allow measurements of a wide range of mechanical configurations with wide range of spring parameters. The device, generally designated 500, is depicted in FIG. 16. Device 500 is configured like a desk comprising horizontal plate 501 mounted between two vertical plates 502. A circular plate 510 (shown in a phantom line) coupled to a strain gage (not shown) for measuring the force applied thereon is centrally located at the frontal part of plate 501 and is covered by a silicon layer to simulate a soft tissue. A spring mounting assembly 520 is slideably mounted on plate 501 for allowing measurements of springs with a wide range of arm lengths. Assembly 520 comprises horizontal plate 521 on which a pole 515 is removeably mounted. In the configuration shown in FIG. 16, two torque springs 530 are coiled around pole 515 having one arm connected to weight 550 and the second arm connected to plate 521 by means of elements 532. The distance between elements 532 can be adjusted to fit the distance between the two ends of the springs. Weight 550 is connected to a stainless cable 560, connected to and wrapped about shaft 555, by means of an adjustable tension pulley 540, comprising two tension pulley wheels 543 and 545. Pulley 540 can be adjusted according to need by adjusting the height of pole 525 mounted between two vertical walls 522 of spring mounting assembly 520. A turning handle 570 provided at one end of shaft 555 allows for arising weight 550 to the desired height above plate 510. A ratchet 575 coupled to turning handle 570 prevents shaft 555 from turning in the opposite direction under the cable tension. Cable 560 is connected to weight 550 by a pull out pin 534 inserted at the top of weight 550. In order to release weight 550, the free end of cable 530 is pulled by pull handle 542 to pull out pin 534, thereby weight 550 is released to hit plate 510. The readings of strain gage are fed into oscilloscope (not shown) calibrated beforehand by placing calibration weights on plate 510. Device 500 was used to measure the force applied by various arrangements of a weight charged by a torque spring, to find arrangements that can be adjusted to deliver a blow of up to about 180 Kg under the constrains of the desired dimensions.

In order to test the force and impact duration generated by a manual pericardial thumping, a group of 28 paramedics (of the Israel MDA organization) trained in CPR emergency, were tested with device 500. The age of the subjects ranges between 20 to 45 years. Physical parameters including height, weight and BMI (body mass index) were obtained for each of the subjects. Measurements were taken for three thumps of each of the subjects on plate 510 to obtain an average thump force per subject. The complete results are summarized in Table 1 appended in drawing sheets 14/15 and 15/15. Statistical analysis of the results gives a median force value of 87.08 Kg. The statistical analysis further reveals that the median force value is almost independent of the physical parameters of the subjects tested. It is therefore assumed that the mechanical defibrillation of the invention should deliver an impact of about 50 to 120 Kg, more preferably of about 70 to 100 KG. However, future tests with a device of the invention are planned for better establishing the optimal parameters of the mechanical impact most suitable for stimulating a cardiac-arrest heart to resume its beating.

It will be appreciated by persons skilled in the art that the present invention is not limited to what has been particularly shown and described hereinabove. Rather the scope of the present invention is defined only by the claims which follow.

1. A mechanical defibrillator comprising an impact member configured to deliver a controllable mechanical impact of a preset force to the chest of a cardiac arrest victim.

2. The mechanical defibrillator of claim 1 wherein said impact member is mounted on a frame adapted to be placed on the victim's chest and wherein the defibrillator further comprises a rechargeable energy storage element coupled to said impact member, said energy storage element is configured to be charged when said impact member is elevated above said frame.

3. The mechanical defibrillator of claim 2 further comprising a discharging mechanism configured for releasing energy stored in the energy storage element into impact energy of said impact member.

4. The mechanical defibrillator of claim 2 further including a charging mechanism for controllably charging said energy storage element while simultaneously elevated said impact member to a controllable height above the chest of the victim.

5. The device of claim 1 wherein said preset force is of a fixed value.

6. The device of claim 6 wherein said fixed value is in the range of 70 to 100 Kg.

7. The device of claim 1 wherein said preset force can be adjusted to up to 180 Kg.

8. The defibrillator of claim 1 further comprising an indicator for indicating said preset force.

9. The defibrillator of claim 1 further comprising at least one sensor for sensing heart's activity of the subject.

10. The defibrillator of claim 7 further comprising analyzing means to analyzing said heart activity and to detect ventricular fibrillation.

11. The defibrillator of claim 8 further comprising halting means to prevent discharging if ventricular fibrillation is not detected.

12. The defibrillator of claim 4 wherein the charging mechanism is manually operated.

13. The defibrillator of claim 3 further comprising activating elements for manually activating said discharging mechanism.

14. The defibrillator of claim 13 wherein said activating elements comprises a discharger element configured to release the impact member, a stopper element configured to prevent said discharging member from discharging the impact element and a stopper releasing element configured to release said stopper element for enabling discharging.

15. The defibrillator of claim 2 wherein said frame further comprises two handles to be held by both hands of an operator of the device.

16. The defibrillator of claim 3 further comprising a main stopper configured to prevent discharging unless the defibrillator is pressed properly against the subject's chest.

17. The defibrillator of claim 2 wherein said rechargeable energy storage element is a mechanical energy storage element.

18. The defibrillator of claim 3 wherein said rechargeable energy storage element is a spring.

19. The defibrillator of claim 3 wherein said rechargeable energy storage element is a torque spring.

20. The defibrillator of claim 1 wherein said impact member is a cylindrical weight of about 100-400 gr and of about 20-50 mm diameter.

21. A mechanical defibrillator for delivering a mechanical impact of a preset force to a sulbject's chest, the device comprising:
a frame adapted to be placed on a subject's chest;
an impact weight mounted on said frame configured to be elevated above the subject's chest;
a chargeable element coupled to said impact element;
a charging mechanism for charging said chargeable element and for elevating said impact weight; and
a discharging mechanism for discharging energy stored in said chargeable element to actuate said impact weight to deliver a mechanical impact to the subject's chest.

22. A method for delivering a defibrillation shock to a cardiac arrest victim comprising the step of delivering a controllable mechanical impact of a preset force to the chest of the victim by means of a mechanical device provided with an impact member.
